(19) United States
(12) Patent Application Publication
(10) Pub. No.: US 2026/0184673 A1
(43) Pub. Date: Jul. 2, 2026

(54) A PROCESS FOR PREPARATION OF AN INTERMEDIATE USEFUL FOR PRODUCING GLUFOSINATE OR ISOMER OR ITS SALTS

(71) Applicant: UPL LIMITED, Mumbai (IN)

(72) Inventors: Prashant Vasant Kini, Mumbai (IN); Chandrasekhar Dayal Mudaliar, Mumbai (IN); Ashishkumar Ravindra Mishra, Mumbai (IN); Shivaji Vithoba Patil, Mumbai (IN)

(73) Assignee: UPL LIMITED, Mumbai (IN)

(21) Appl. No.: 19/131,841

(22) PCT Filed: Dec. 8, 2023
(86) PCT No.: PCT/IN2023/051147

§ 371 Date: May 21, 2025

(30) Foreign Application Priority Data
Dec. 12, 2022 (IN) 202221071544

(51) Int. Cl. C07C 269/04; C07F 9/30

(52) U.S. Cl. CPC C07C 269/04; C07F 9/301

(57) ABSTRACT

The present invention relates to a process preparation of a compound of formula (I) O O NHZ R 2 R 1 Formula (I) wherein, R1 is amino protecting group, R2 is carboxylic acid protecting group; and 5 Z is halogen or hydroxy group 10

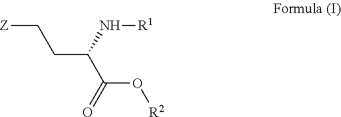

### FIELD OF INVENTION

The present invention relates to a process preparation of a compound of formula (I) which is a key intermediate in synthesis of L-glufosinate or its salt.

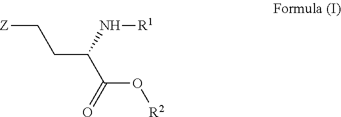

wherein, R¹ is amino protecting group, R² is carboxylic acid protecting group; and Z is halogen or hydroxy group.

### BACKGROUND OF THE INVENTION

Glufosinate is non-selective herbicide belonging to the group of organophosphate herbicides. It is generally used in form of ammonium salt for total vegetation control and to control growth of weeds and grasses. Glufosinate is a racemic mixture of L- and D-glufosinate. However, it is well known that L-glufosinate i.e. (S)-2-amino-4-(hydroxy(methyl)phosphonoyl)butanoic acid) is much more potent than Glufosinate.

There are various methods for preparation of L-glufosinate, one of the convenient methods for preparation of L-glufosinate proceeds through formation of compound of formula (I).

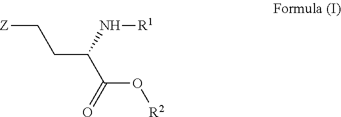

wherein, R¹ is amino protecting group, R² is carboxylic acid protecting group; and Z is halogen or hydroxy group.

CN106083922B discloses a multiple steps process for preparation of L-glufosinate-ammonium, proceeding though preparation of said compound of formula (I), as an intermediate. In the first step, L-methionine is reacted with methyl chloroacetate in presence of tetrabutylammonium bromide and water at 90° C., after completion of reaction the L-homoserine lactone hydrochloride is isolated by workup procedure wherein following steps were followed sequentially
i) the reaction mixture was first extracted with dichloromethane,
ii) then treated with 37% hydrochloric acid,
iii) organic solvent was then removed under reduced pressure
iv) absolute ethyl alcohol was added to residue and mixture cooled to 0° C. to obtain L-homoserine lactone hydrochloride

The L-homoserine lactone hydrochloride thus obtained is reacted with ethyl chloroformate in presence of water and sodium carbonate at 0-5° C. to yield N-protected L-homoserine lactone, this compound is isolated by carrying out following steps
i) reaction mixture was treated with hydrochloric acid to adjust pH to 5,
ii) the solvent was then removed by evaporation
iii) residue was treated with acetone,
iv) sodium chloride formed as by product was removed by filtration,
v) later acetone removed under reduced pressure and
vi) the product obtained was recrystallised by anhydrous ethanol.

N-protected L-homoserine lactone thus obtained is reacted with ethanol and thionyl chloride in presence of zinc chloride at 50° C. to get compound of formula (I), wherein isolation is caried out by following steps
i) the solvent used was evaporated
ii) 1, 2-dichloroethane and water was added
iii) the layers were separated
iv) the water layer was extracted with dichloroethane for 2 times,
v) organic layers were combined, and the solvent was removed under reduced pressure

Subsequent reaction of compound of formula (I) provides L-glufosinate ammonium.

The above process requires tedious multistep workup procedure to isolate intermediate at each step, thereby reducing the overall yield of the product. Furthermore, use of multiple solvents for each step leads to effluent generation and additional cost for separating these solvents for reuse, thereby making the process uneconomical and environmentally unfavourable.

To overcome these problems associated with in the art the inventors of present invention have developed a novel and simple process for preparing intermediates useful for preparing glufosinate or isomer or salts thereof. The present process avoids multiple isolation and purification steps of each intermediate and thereby reducing the number of steps of the process and improving efficiency of the reaction by providing product in high yield.

### OBJECT OF THE INVENTION

The main object of the present invention is to provide an economical and environmentally friendly process for preparation of a compound of formula (I).

Another object of present invention is to provide a simple and industrially feasible process for preparation of compound of formula (I) starting from compound of formula (II) without isolation/purification of intermediates.

Yet another object and advantage of present invention is to provide a process for preparation of a compound of formula (I) generating low volumes of effluent/s.

Another object of present invention is to provide a simple and scalable process for preparation of compound of formula (I).

### SUMMARY OF THE INVENTION

According to an aspect of the present invention a process for preparation of a compound of formula (I)

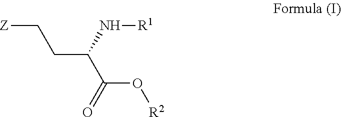

wherein, R¹ is amino protecting group, R² is carboxylic acid protecting group; and Z is halogen or hydroxy group;
the process comprising converting L-homoserine lactone of formula (IV) or its salt, to compound of formula (I):

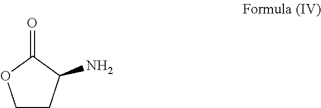

wherein the compound of formula (IV) is substantially free of a compound of formula (V):

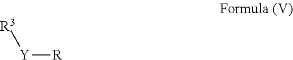

wherein:
R is hydrogen, alkyl, aryl, acyl, alkylsulfonate esters, alkylnitrate ester, alkylphosphoryl or alkylsilyl;
Y is selected from O, N or S; and
R³ is hydrogen or R⁴—O—C(O)—CH₂—.

According to an aspect of the present invention a process for preparation of a compound of formula (I)

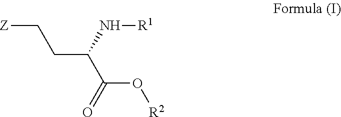

wherein, R¹ is amino protecting group, R² is carboxylic acid protecting group; and
Z is halogen or hydroxy group
the steps comprising:
   a) reacting compound of formula (II) with compound of formula (III) to obtain L-homoserine lactone of formula (IV) or its salt; and a by-product of formula (V)

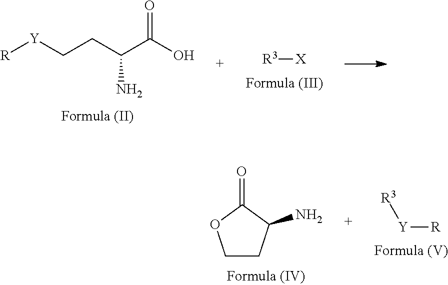

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V)
c) converting L-homoserine lactone of formula (IV) or its salt, to compound of formula (I)

In an aspect, the present invention provides a compound of formula (IV) being substantially free of a compound of formula (V), prepared by removing the compound of formula (V) from the reaction product obtained by reacting compound of formula (II) with compound of formula (III);

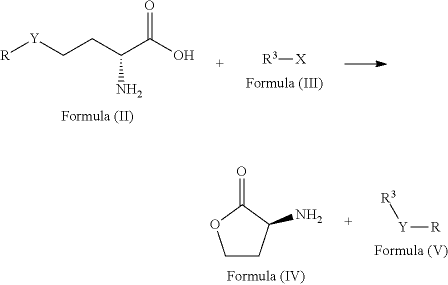

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group;
X is halogen, —HSO₄, —H₂PO₄; and
Y is selected from O, N or S.

According to an aspect of the present invention, there is provided a process for preparation of a compound of formula (I)

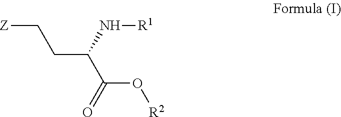

wherein, R¹ is amino protecting group, R² is carboxylic acid protecting group; and
Z is halogen or hydroxy group
the steps comprising:
   a) reacting compound of formula (II) with compound of formula (III) to obtain L-homoserine lactone of formula (IV) or its salt; and a by-product of formula (V)

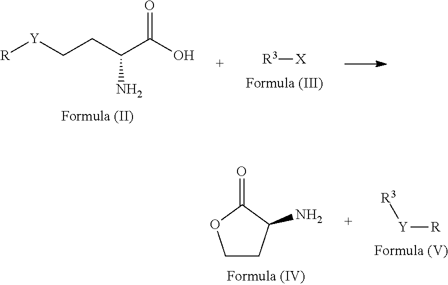

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V),
c) converting L-homoserine lactone of formula (IV) or its salt, to compound of formula (I)
wherein step c) comprises protecting the amino group of the L-homoserine lactone of formula (IV) obtain compound of formula (VI) and conversion of compound of formula (VI) to compound of formula (I).

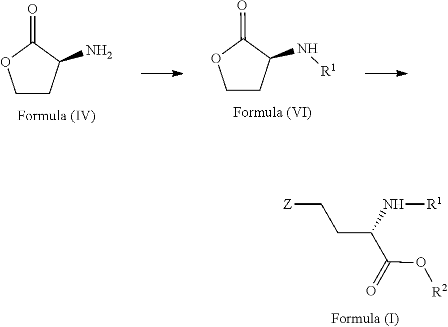

According to an aspect of the present invention, there is provided a process for preparation of a compound of formula (I)

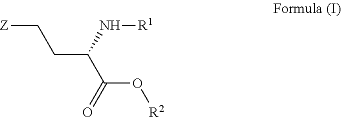

wherein, R¹ is amino protecting group, R² is carboxylic acid protecting group; and
Z is halogen or hydroxy group,
comprising the steps of:
   a) reacting compound of formula (II) with compound of formula (III) to obtain L-homoserine lactone of formula (IV) or its salt; and a by-product of formula (V)

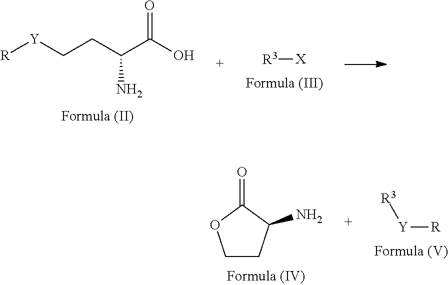

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V)
c) converting L-homoserine lactone of formula (IV) or its salt, to compound of formula (I)
wherein step c) comprises protecting the amino group of the L-homoserine lactone of formula (IV) or its salt, obtain compound of formula (VI) and conversion of compound of formula (VI) to compound of formula (I);

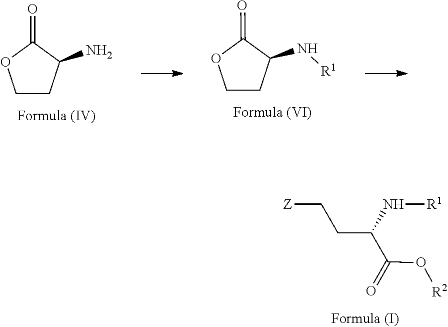

and wherein the process proceeds without isolation or purification of L-homoserine lactone of formula (IV) or its salt, and compound of formula (VI).

According to an aspect of the present invention, there is provided a process for preparation of L-glufosinate or its salts using said compound of formula (I) prepared by present process.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

As used in the specification and the claims, the singular forms “a”, “an”, and “the” include plural referents unless the context clearly dictates otherwise.

The present disclosure is not to be limited in scope by the specific embodiments described herein, which are intended for the purposes of exemplification only.

The term “room temperature” unless stated otherwise, essentially means temperature in range of 20-35° C.

The term “purity” means purity as determined by GC (“Gas chromatography”).

The term “about” shall be interpreted to mean “approximately” or “reasonably close to” and any statistically insignificant variations therefrom.

As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

The term “amino-protecting group” as used herein refers to a substituent that protects an amino functionality against undesirable reactions during synthetic procedures. Amino-protecting groups are typically selected from acyl group such as C(═O)Y, wherein Y can be OR′ or R′, wherein R′ is substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; or it can be urea, urethane, nitroso, nitro, sulphenyl, sulphonyl, sulfonic acid, or trialkylsilyl. Examples include acetyl, carbobenzyloxy (also benzyloxycarbonyl or carbobenzoxy), formyl, t-butyloxycarbonyl, fluorenylmethyloxycarbonyl, 2-nitrophenylsulfenyl, methanesulfonyl, p-toluenesulfonyl, and the like.

The term “carboxylic acid protecting group” as used herein refers to a substituent that protects carboxyl functionality against undesirable reactions during synthetic procedures. The carboxylic acid protecting groups are typically selected from alkyl group substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group.

The term “Hydroxy group” used herein refers to hydroxy group or its derivatives. “Alkyl” as used herein means a straight or branched chain saturated aliphatic hydrocarbon having the specified number of carbon atoms, specifically 1 to 10 carbon atoms, more specifically 1 to 8 carbon atoms.

The term “Halogen” as used herein means a fluorine, chlorine, bromine or iodine atoms.

The term “Alkenyl” as used herein means a straight or branched chain unsaturated aliphatic hydrocarbon with carbon-carbon double bond having the specified number of carbon atoms, specifically 2 to 10 carbon atoms.

The term “Alkynyl” refers to an alkyl group containing at least one carbon-carbon triple bond and having from 2 to 10 carbon atoms. The term “alkynyl” also includes those groups having one triple bond and one double bond.

The term “Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, and the likes

The term “Aryl” refers to an aromatic carbocyclic group having a single ring (e.g. monocyclic) or multiple rings (e.g. bicyclic or tricyclic) including fused systems. Examples of aryl groups include phenyl, naphthyl, and the likes.

The term “Acyl” refers to C(═O)R′, and wherein R′ is substituted/unsubstituted C₁ to C₁₀ alkyl group. Examples of acyl group is acetyl group and the likes.

The term “substantially free of compound of formula (V)” means the percentage of compound of formula (V) is less than 5%, preferably less than 4%, preferably less than 3%, preferably less than 2%, preferably less than 1%, more preferably less than 0.5%, in compound of formula (IV).

The term “L-glufosinate” shall be interpreted to mean L-glufosinate or its salts. The salts of L-glufosinate such as monosodium salt, disodium salt, monopotassium salt, dipotassium salt, calcium salt, ammonium salt, —NH3(CH3)+ salt, —NH2(CH3)2+ salt, —NH(CH3)3+ salt, —NH(CH3)2(C2H4OH)+ salt, and —NH2(CH3)(C₂H4OH)+ salt are included in the definition. The agronomically acceptable salts include L-glufosinate-ammonium, L-glufosinate-sodium, and L-glufosinate-potassium.

According to an aspect of the present invention a process for preparation of a compound of formula (I)

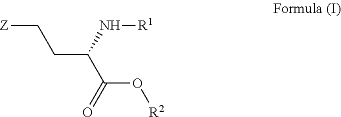

wherein, R¹ is amino protecting group, R² is carboxylic acid protecting group; and Z is halogen or hydroxy group;
the process comprising converting L-homoserine lactone of formula (IV) or its salt, to compound of formula (I):

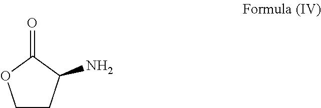

wherein the compound of formula (IV) is substantially free of a compound of formula (V):

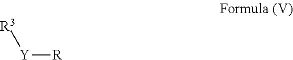

wherein:
R is hydrogen, alkyl, aryl, acyl, alkylsulfonate esters, alkylnitrate ester, alkylphosphoryl or alkylsilyl;
Y is selected from O, N or S; and
R³ is hydrogen or R⁴—O—C(O)—CH₂—.

According to another aspect of the present invention, there is provided a process for preparation of a compound of formula (I)

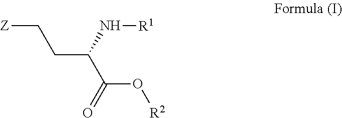

wherein, R¹ is amino protecting group, R² is carboxylic acid protecting group; and
Z is halogen or hydroxy group
the steps comprising:
   a) reacting compound of formula (II) with compound of formula (III) to obtain L-homoserine lactone of formula (IV) or it salt; and a by-product of formula (V)

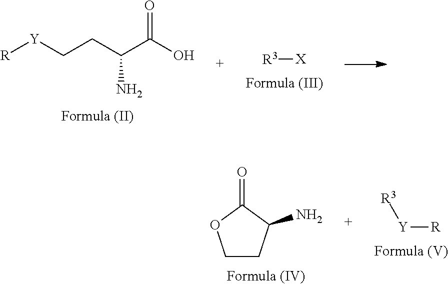

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V)
c) converting L-homoserine lactone of formula (IV) or its salt, to compound of formula (I).

In an embodiment the formula (I) represents a compound wherein R¹ is amino protecting group selected from acyl group such as C(═O)A, wherein A can be OR′ or R′, wherein R′ is substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; or it can be urea, urethane, nitroso, nitro, sulphenyl, sulphonyl, sulfonic acid, or trialkylsilyl; R² is carboxylic acid protecting group selected from substituted or unsubstituted C₁ to C₁₀ alkyl group or a substituted or unsubstituted C₆ to C₂₀ aryl group; Z is halogen or hydroxy group.

In an embodiment, the formula (I) represents a compound wherein R¹ is amino protecting group selected from acyl group such as C(═O)A, wherein A can be OR′ or R′, wherein R′ is C₁ to C₅ alkyl; R² is carboxylic acid protecting group selected from substituted or unsubstituted C₁ to C₅ alkyl group; and Z is halogen.

In an embodiment, the formula (II) represents a compound wherein Y is selected from O, N or S; R is selected from hydrogen, C₁ to C₅ alkyl or an acyl group of formula —C(═O)R′, and wherein R′ is C₁ to C₅ alkyl.

In an embodiment, the formula (II) represents a compound wherein Y is S and R is C₁ to C₅ alkyl.

In an embodiment, the formula (II) compound is L-methionine.

In an embodiment, the formula (II) represents a compound wherein Y is O; and R is hydrogen.

In an embodiment, the formula (II) compound is L-homoserine.

In an embodiment, the formula (II) represents a compound wherein Y is O; and R is acyl group of formula —C(═O)R′, and wherein R′ is C₁ to C₅ alkyl.

In an embodiment, the formula (II) compound is O-acetyl L-homoserine.

In an embodiment, the formula (III) represents a compound wherein R³ is hydrogen or R⁴—O—C(O)—CH₂—; R⁴ is selected from hydrogen or C₁ to C₅ alkyl group; and X is halogen.

In an embodiment, the formula (III) represents a compound wherein R³ is hydrogen and X is halogen.

In an embodiment, the formula (III) represents a compound wherein R³ is hydrogen and X is chlorine.

In an embodiment, the formula (III) represents a compound wherein R³ is R⁴—O—C(O)—CH₂—; R⁴ is selected from hydrogen or C₁ to C₅ alkyl group; and X is halogen.

In an embodiment, formula (III) represents any mineral acid, including hydrochloric acid, hydroiodic acid, hydrobromic acid, sulfuric acid, phosphoric acid, polyphosphoric acid and the like.

In an embodiment, step a) is carried out in presence of an acid catalyst when the formula (III) represents a compound wherein R³ is R⁴—O—C(O)—CH₂—; R⁴ is selected from hydrogen or C₁ to C₅ alkyl group; and X is halogen.

The acid catalyst used is selected from, but not limited to, p-toluene sulfonic acid, methane sulfonic acid, benzene sulfonic acid, hydrochloric acid, phosphoric acid, sulfuric acid, trichloroacetic acid, trifluoroacetic acid, and the likes.

The amount of acid catalyst used is in the range of 0 to 0.1 moles with respect to compound of formula (II).

In an embodiment, the step a) of the process is carried out in presence of a solvent.

The solvent used is selected from, but not limited to, water or an organic solvent selected from, but not limited to, C₁-C₅ alcohols such as methanol, ethanol, isopropanol, n-propanol; ketones like acetone; esters such as ethyl acetate; chlorinated solvents like dichloromethane or mixture thereof.

The weight ratio of solvent used in step a) with respect to compound of formula (II) is in the range of about 0.1 to 3: 1.

In an embodiment, the step a) of the process is carried out at temperature ranging from 0° C. to 120° C.

In an embodiment, the step a) of the process is carried out in presence of a phase transfer catalyst.

The phase transfer catalyst used may be selected from quaternary ammonium salt such as tetrabutylammonium chloride, tetrabutylammonium bromide, tetrabutylammonium iodide, benzyltriethylammoinium chloride and the like.

In an embodiment, the step a) of the process is carried out in absence of a phase transfer catalyst.

In an embodiment, after completion of reaction of step a), the reaction mixture comprising L-homoserine lactone of formula (IV) or its salt, and a by-product of formula (V) is obtained.

In an embodiment, L-homoserine lactone of formula (IV) is in form of salt.

In an embodiment, L-homoserine lactone of formula (IV) in form of salt can be represented as formula (IVa) as follows

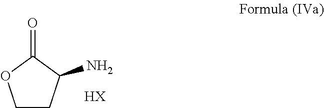

wherein X is halogen.

In an embodiment, in the step b) of the process the by-product of formula (V) is removed from the reaction mixture of step a).

In an embodiment, the by-product of formula (V) is removed from the reaction mixture in step b) using the conventional methods such as distillation, azeotropic distillation, solvent extraction followed by distillation, chemical treatment and the like.

In an embodiment, the by-product of formula (V) is removed by distillation.

In an embodiment, the by-product of formula (V) is removed by azeotropic distillation.

In another embodiment, the by-product of formula (V) is removed by extracting the reaction mixture of step a) with a water-immiscible solvent.

The water-immiscible solvent used is selected from, but not limited to, a chlorinated solvent such as dichloroethane, dichloromethane, chloroform and the likes; an ester solvent such as ethyl acetate, isopropyl acetate, isobutyl acetate and the likes; ethers such as methyl tert-butyl ether (MTBE) and the likes; ketones, such as 2-butanone and the likes; hydrocarbons such as cyclohexane, cycloheptane, toluene, xylene and the likes.

In an embodiment, after removal of compound of formula (V) from the reaction mixture in step b), the reaction mixture is treated with an inorganic acid. The inorganic acid such as hydrochloric acid, hydrobromic acid, hydroiodic acid, sulfuric acid, nitric acid and the likes.

In an embodiment, after removal of compound of formula (V) from the reaction mixture, the moisture content of the reaction mixture is adjusted below 12%.

In an embodiment, the moisture content of the reaction mixture was reduced using conventionally known methods such as distillation.

In an embodiment, after removal of compound of formula (V) the pH of the reaction mixture is adjusted to 3.5 to 6.5.

In an embodiment, step c) comprises protection of the amino group of the L-homoserine lactone of formula (IV) or its salt, to obtain compound of formula (VI) followed by conversion to compound of formula (I).

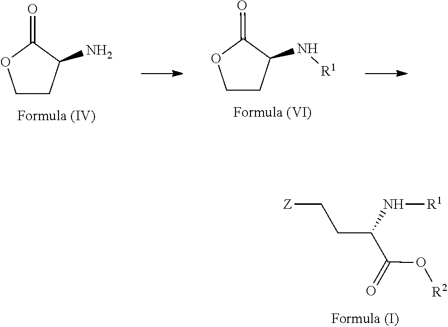

In an embodiment, the process of present invention proceeds without isolation or purification of the intermediates formed in any of the steps of the process.

In an embodiment, the compound of formula (VI) obtained in step c) is not isolated and the reaction mixture comprising compound of formula (VI) is used in next step.

In an embodiment, the reaction mixture comprising compound of formula (VI) is extracted with a suitable organic solvent.

The organic solvent used is selected from chlorinated solvents such as dichloromethane, dichloroethane, chloroform and the likes; hydrocarbons such as cyclohexane, toluene, xylene and the likes; alcohols such as methanol, ethanol, n-propanol, isopropanol and the likes; esters such as ethyl acetate, isobutyl acetate and the likes; ethers such as methyl tert-butyl ether (MTBE) and the likes; ketones, such as 2-butanone and the likes; preferably chlorinated solvents such as dichloromethane, dichloroethane, chloroform and the likes; or hydrocarbon such as cyclohexane, toluene, xylene and the like are used.

In an embodiment, the organic solvent extract comprising compound of formula (VI) is reduced to 30 to 80% of total volume by distillation.

In an embodiment, the organic solvent in the extract obtained is recovered by distillation and recycled.

According to an aspect of the present invention, there is provided a process for preparation of a compound of formula (I)

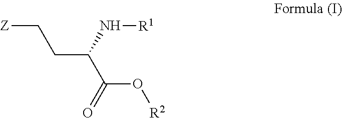

wherein, R¹ is amino protecting group, R² is carboxylic acid protecting group; and
Z is halogen or hydroxy group
the steps comprising:
   a) reacting compound of formula (II) with compound of formula (III) to obtain L-homoserine lactone of formula (IV) or its salt; and a by-product of formula (V)

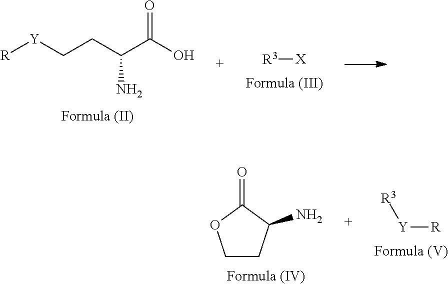

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V)
c) converting L-homoserine lactone of formula (IV) or its salt, to compound of formula (I)
wherein step c) comprises protection of amino group of the L-homoserine lactone of formula (IV) or its salt, to obtain compound of formula (VI) followed by conversion to compound of formula (I).

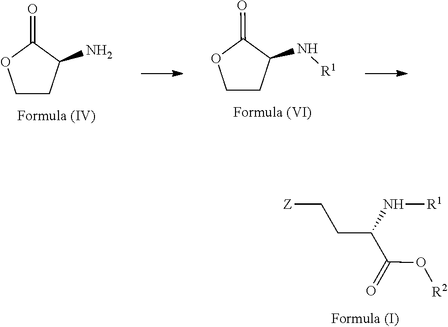

In an embodiment, in the step c), the amino protecting group used for protecting the amino group of the L-homoserine lactone of formula (IV) is selected from group comprising of acyl group such as C(═O)Y, wherein Y can be OR or R, wherein R is substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; or urea, urethane, nitroso, nitro, sulphenyl, sulphonyl, sulfonic acid, or trialkylsilyl. Examples include acetyl, carbobenzyloxy (also benzyloxycarbonyl or carbobenzoxy), formyl, t-butyloxycarbonyl, fluorenylmethyloxycarbonyl, 2-nitrophenylsulfenyl, methanesulfonyl, p-toluenesulfonyl, p-nitro phenyl sulfonyl group and the like.

Preferably, the amino protecting group is acyl group such as C(═O)Y, wherein Y can be OR or R, wherein R is substituted or unsubstituted C₁ to C₁₀ alkyl group.

In an embodiment, said protection in step c) is carried by treating L-homoserine lactone of formula (IV) or its salt, with ethylchloroformate to obtain compound of formula (VI).

In an embodiment, said protection in step c) is carried by treating salt of L-homoserine lactone of formula (IVa) with ethylchloroformate to obtain compound of formula (VI).

In an embodiment, said protection in step c) is carried out in a biphasic solvent system.

The biphasic solvent system comprises of water and at least one water-immiscible solvent. The water-immiscible solvent used may be selected from the group including, but not limited to, dichloromethane, dichloroethane, chloroform, carbon tetrachloride, diethyl ether, ethyl acetate, hexane, cyclohexane, heptane, methyl-tert-butyl ether toluene, benzene, and the like.

In an embodiment, the biphasic solvent system comprises of water and dichloroethane.

In an embodiment, the biphasic solvent system comprises of water and ethyl acetate.

In an embodiment, the weight ratio of biphasic solvent system and compound of formula (II) is in the range of about 1 to 6:1.

In an embodiment, said protection in step c) is carried out at temperature ranging from −10 to 10° C.

In an embodiment, in the step c) the compound of formula (VI) is converted to compound of formula (I) by treatment with a halogenating agent and an alcohol.

In an embodiment, the compound of formula (VI) is converted to compound of formula (I) by treatment with halogenating agent and an alcohol, wherein halogenating agent and alcohol are added sequentially or simultaneously.

In an embodiment, the sequential addition of halogenating agent and an alcohol, is in any order.

The halogenating agent used may be selected from the group comprising of, but not limited to, chlorine, phosphorus trichloride (PCl₃), phosphorus pentachloride (PCl₅), phosphorus oxychloride (POCl₃), thionyl chloride, sulfuryl chloride, oxalyl chloride, sulfonyl chloride, acetyl chloride, phosgene, bromine, phosphorus tribromide (PBr₃), boron tribromide (BBr₃), Iodine, phosphorus triiodide (PI₃), N-chlorosuccinimide, and N-bromosuccinimide.

The alcohol used may be selected from the group comprising of, but not limited to, C₁-C₈ alcohols like methanol, ethanol, isopropanol, n-propanol, butanol, tert-butanol, benzyl alcohol and the like.

In an embodiment, said conversation of the compound of formula (VI) to compound of formula (I) is carried out in presence of a solvent.

The solvent used may be selected from the group including, but not limited to, nitriles like acetonitrile or chlorinated solvent like dichloromethane, dichloroethane, chloroform, 1,1,2-trichloroethane, chlorobenzene, and dichlorobenzene.

In an embodiment, said conversation of the compound of formula (VI) to compound of formula (I) is carried out at temperature ranging from 0 to 50° C. for 0.5 to 20 hours.

In an embodiment, the solvents used in the process of the present invention are recovered and recycled.

In an embodiment, the present invention provides a process for preparation of a compound of formula (I), wherein there is a low solvent/s consumption and low volume effluent generation.

According to an embodiment, the reaction in general can be is illustrated by scheme I which follows:

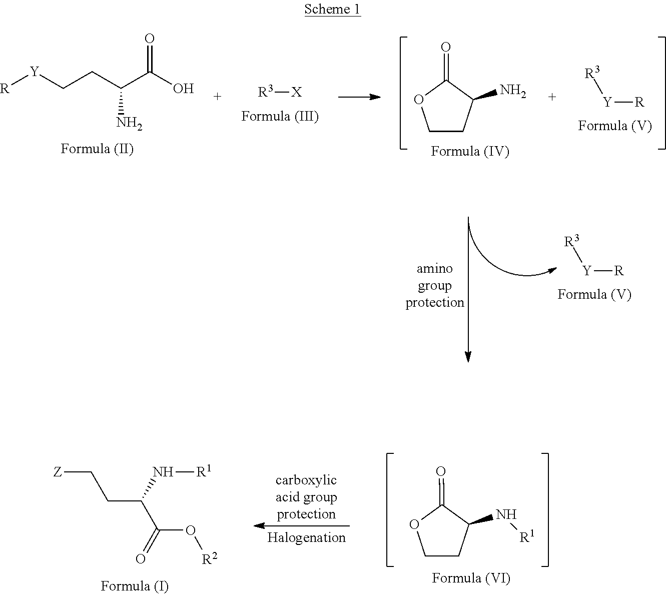

R is hydrogen, alkyl, aryl, acyl, alkylsulfonate esters, alkynitrate ester, alkylphosphoryl or alkylsilyl;
R¹ is amino protecting group,
R² is carboxylic acid protecting group;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
Z is halogen or hydroxy group

According to an aspect of the present invention, there is provided a process for preparation of a compound of formula (I)

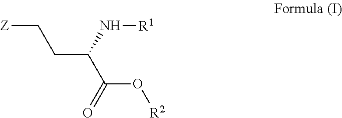

wherein, R¹ is amino protecting group, R² is carboxylic acid protecting group; and
Z is halogen or hydroxy group
the steps comprising:
   a) reacting compound of formula (II) with compound of formula (III) to obtain L-homoserine lactone of formula (IV) or its salt; and a by-product of formula (V)

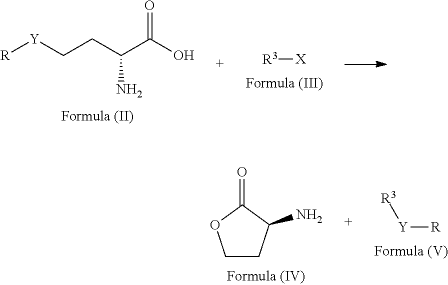

wherein,
R is hydrogen, alkyl, aryl, acyl, alkylsulfonate esters, alkylnitrate ester, alkylphosphoryl or alkylsilyl;
R³ is hydrogen or R⁴—O—C(O)—CH₂—
R⁴ is selected from hydrogen, C₁ to C₅ alkyl group; and
X is halogen, —HSO₄, —H₂PO₄
Y is selected from O, N or S;
b) removing the by-product of formula (V)
c) converting L-homoserine lactone of formula (IV) or its salt, to compound of formula (I),
wherein step c) includes protecting the amino group of the L-homoserine lactone of formula (IV) or its salt, obtain compound of formula (VI) and conversion of compound of formula (VI) to compound of formula (I);

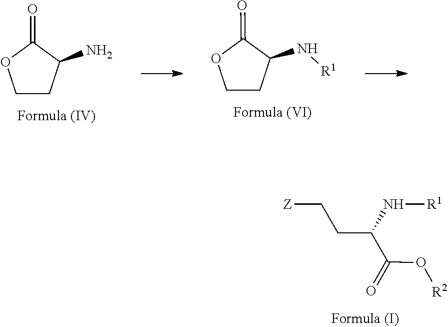

and wherein the process proceeds without isolation or purification of L-homoserine lactone of formula (IV) or its salt, and compound of formula (VI).

In an embodiment, after completion of reaction of step a), the reaction mixture comprising L-homoserine lactone of formula (IV) or its salt, and a by-product of formula (V) is obtained.

In an embodiment, in the step b) the by-product of formula (V) is removed by conventionally known methods; and the reaction mixture comprising compound of formula (IV) or its salt, is used in step c).

In an embodiment, the reaction mixture of step b) comprises L-homoserine lactone of formula (IV) is in form of salt.

In an embodiment, the process of present invention proceeds without isolation or purification of the intermediates formed in any of the steps of the process.

In an embodiment, the process of present invention proceeds without isolation or purification of the compound of formula (IV) or its salt, and/or the compound of formula (VI) formed as intermediates in the process.

The process of the present invention obviates isolation and/or purification of the compound of formula (IV) or its salt, and/or compound of formula (VI) thereby reducing the number of operations and saving time. The process is particularly advantageous as it makes possible to produce highly pure compound of formula (I) in a simple and cost-effective manner, which was not the case of the known processes.

In an embodiment, compound of formula (I) can be presented as compound of formula (Ia) wherein R¹ is acyl group such as C(═O)A, wherein A is OR′, and R′ is ethyl group; R² is ethyl group; Z is chlorine; and the process of present invention can be illustrated by scheme II which follows:

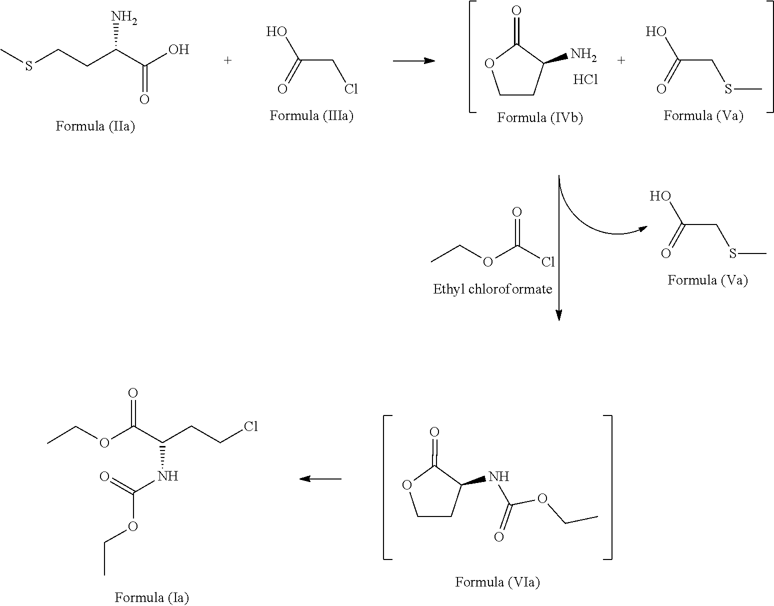

In the scheme (II) provided above, compound of formula (IIa) is compound of formula (II) wherein Y is S and R is methyl group; compound of formula (IIa) is compound of formula (III) wherein R³ is R⁴—O—C(O)—CH₂—, wherein R⁴ is hydrogen, and X is chlorine; compound of formula (IVb) is compound of formula (IV) in salt form; compound of formula (Va) is compound of formula (V) wherein Y is S, R³ is R⁴—O—C(O)—CH₂—, wherein R⁴ is hydrogen and R is methyl group; and compound of formula (VIa) is compound of formula (VI) wherein R¹ is acyl group such as C(═O)A, wherein A is OR′, and R′ is ethyl group.

In another embodiment, compound of formula (I) can be presented as compound of formula (Ia) wherein R^{F} is acyl group such as C(═O)A, wherein A is OR′, and R′ is ethyl group; R² is ethyl group; Z is chlorine; and the process of present invention can be illustrated by scheme III which follows:

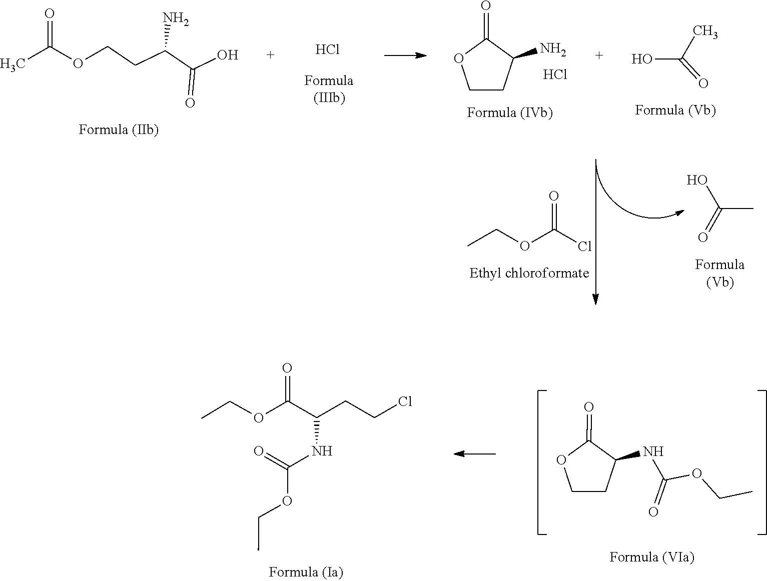

In the scheme (III) provided above, compound of formula (IIb) is compound of formula (II) wherein Y is O and R is acyl group of formula —C(═O)R′, and wherein R′ is methyl group; compound of formula (IIIb) is compound of formula (III) wherein R³ is hydrogen, and X is chlorine; compound of formula (IVb) is compound of formula (IV) in salt form; compound of formula (Vb) is compound of formula (V) wherein Y is O, R³ is hydrogen, and R is acyl group of formula —C(═O)R′, and wherein R′ is methyl group; and compound of formula (VIa) is compound of formula (VI) wherein R1 is acyl group such as C(═O)A, wherein A is OR′, and R′ is ethyl group.

In another embodiment, compound of formula (I) can be presented as compound of formula (Ia) wherein R¹ is acyl group such as C(═O)A, wherein A is OR′, and R′ is ethyl group; R² is ethyl group; Z is chlorine; and the process of present invention can be illustrated by scheme IV which follows:

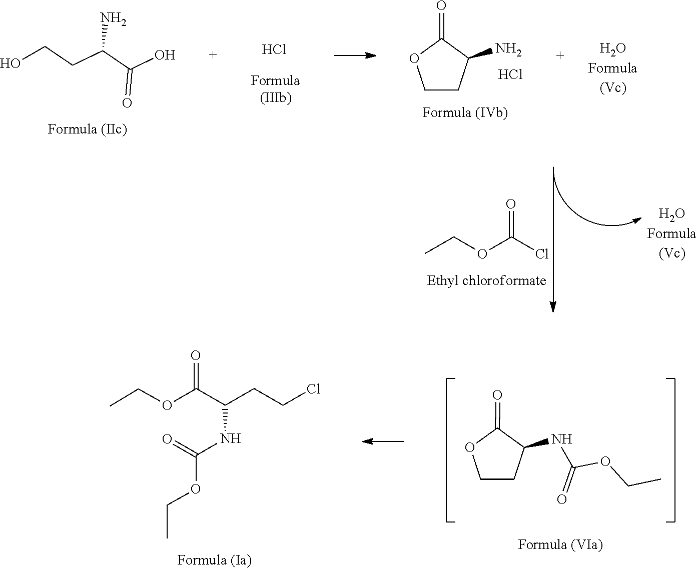

In the scheme (IV) provided above, compound of formula (IIc) is compound of formula (II) wherein Y is O and R is hydrogen; compound of formula (IIIb) is compound of formula (III) wherein R³ is hydrogen, and X is chlorine; compound of formula (IVb) is compound of formula (IV) in salt form; compound of formula (Vc) is compound of formula (V) wherein Y is O, R³ is hydrogen, and R is hydrogen; and compound of formula (VIa) is compound of formula (VI) wherein R1 is acyl group such as C(═O)A, wherein A is OR′, and R′ is ethyl group.

In an embodiment, the compound of formula (I) prepared according to present invention is used for preparation of L-glufosinate or its salts.

In another embodiment, the compound of formula (I) prepared according to present invention is used in process for preparation of L-glufosinate or its salts, by methods known in prior art.

In an aspect the present invention provides a process for preparation of glufosinate or salts thereof using a compound of formula (I) prepared according to the present invention.

In an aspect the present invention provides a process for preparation of L-glufosinate using a compound of formula (I) prepared according to the present invention.

In an embodiment, the present invention provides a process for preparing a L-glufosinate or salts thereof, wherein the process comprising converting a compound of formula (I) to L-glufosinate or salts thereof, wherein preparation of compound of formula (I) comprising converting L-homoserine lactone of formula (IV) or its salt, to compound of formula (I):

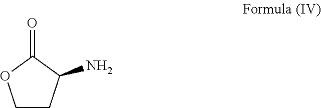

wherein the compound of formula (IV) is substantially free of a compound of formula (V):

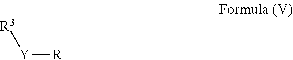

wherein:
R is hydrogen, alkyl, aryl, acyl, alkylsulfonate esters, alkylnitrate ester, alkylphosphoryl or alkylsilyl;
Y is selected from O, N or S; and
R³ is hydrogen or R⁴—O—C(O)—CH₂—.

In an embodiment, the present invention provides a process for preparing L-glufosinate or its salts comprising a compound of formula (I),

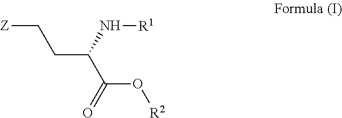

wherein, R¹ is amino protecting group, R² is carboxylic acid protecting group; and Z is halogen or hydroxy group;
prepared by a process comprising converting L-homoserine lactone of formula (IV) or its salt, to compound of formula (I):

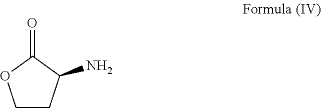

wherein the compound of formula (IV) is substantially free of a compound of formula (V):

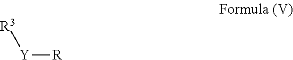

wherein:
R is hydrogen, alkyl, aryl, acyl, alkylsulfonate esters, alkylnitrate ester, alkylphosphoryl or alkylsilyl;
Y is selected from O, N or S; and
R³ is hydrogen or R⁴—O—C(O)—CH₂—.

In an embodiment the yield of the compound of formula (I) is enhanced by at least 5% wherein the compound of formula (I) is prepared by the present invention.

### Advantages of the Invention

1. A simple, cost-effective and industrially viable process for preparation of a compound of formula (I).
2. Said process does not require isolation/purification of compound of Formula (IV) or (VI), thereby reducing effluent generation and the number of steps of the operations.
3. The solvents used in the process of present invention can be easily recovered and recycled.
4. The overall yield of compound of formula (I) obtained by process of present invention is increased at least by 15%.
5. The compound of formula (I) obtained by process of present invention has high purity.

### EXAMPLES

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### Analytical Method Details:

Samples were analysed on High Performance Liquid Chromatograph with UV detector and integrator/software at 195 nm using Inertsil—C18 (250×4.6 mm i.d., micron); and on gas liquid chromatograph with flame ionization detector (FID) using DB-5 (30 meter length, 0.25 mm id, 0.25 um film thickness).

### Example 1: Preparation of Ethyl (2S)-4-chloro-2-[(ethoxycarbonyl)amino]butanoate [Compound of Formula Ia]

(with Phase Transfer Catalyst)

A mixture of 249.5 g of chloroacetic acid, 125.6 ml of methanol and 5 g of p-toluene sulfonic acid was heated to 55-60° C. for 2 hours, followed by addition of 375 ml of water, 300 g of L-methionine and 2.20 g of tetramethyl ammonium chloride. The reaction mixture thus obtained was heated to 90-95° C. and maintained at this temperature for 6-7 hours. The mixture was then cooled and extracted with ethylene dichloride The aqueous layer was collected and treated with 215.5 ml of 30% HCl, the mixture was then subjected to distillation under vacuum at 60-70° C. till moisture content was below 3-5%. To the mixture was added 600 ml of water was added. The pH of aqueous layer obtained was adjusted to 4 to 4.5 using aqueous solution of sodium carbonate and subsequently, 480 ml of ethylene chloride was added to it. To this mixture was then added simultaneously, sodium carbonate solution (prepared from 326 g of sodium carbonate and 600 ml of water) and a solution of ethyl chloroformate (prepared from 172 ml of ethyl chloroformate and 300 ml of ethylene dichloride) at 0 to −5° C. in 3 to 4 hours. The layers were separated, and the organic layer obtained was partially reduced by distillation under vacuum to obtain a reaction mass. To the reaction mass was then added 375.6 ml of ethanol and 250 ml of thionyl chloride in 6 hours. The mixture was maintained at room temperature for 12 hours until product formation is completed. The solvent used was recovered by distillation to obtain 398 g of ethyl (2S)-4-chloro-2-[(ethoxycarbonyl)amino]butanoate having wt/wt purity of 94%. Overall Yield 78.35%

### Example 2: Preparation of Ethyl (2S)-4-chloro-2-[(ethoxycarbonyl)amino]butanoate [Compound of Formula Ia]

(without Using Phase Transfer Catalyst)

A mixture of 249.5 g of chloroacetic acid, 125.6 ml of methanol and 5 g of p-toluene sulfonic acid was heated to 55-60° C. for 2 hours, followed by addition of 375 ml of water and 300 g of L-methionine. The reaction mixture thus obtained was heated to 90-95° C. and maintained at this temperature for 6-7 hours. The mixture was then cooled and extracted with ethylene dichloride. The aqueous layer was collected and treated with 215.5 ml of 30% HCl, the mixture was then subjected to distillation under vacuum at 60-70° C. till moisture content was below 3-5%. To the mixture was then added 600 ml of water. The pH of aqueous layer obtained was adjusted to 4 to 4.5 using aqueous solution of sodium carbonate and subsequently, 480 ml of ethylene chloride was added to it. To this mixture was then added simultaneously, sodium carbonate solution (prepared from 326 g of sodium carbonate and 600 ml of water) and a solution of ethyl chloroformate (prepared from 172 ml of ethyl chloroformate and 300 ml of ethylene dichloride) at 0 to −5° C. in 3 to 4 hours. The layers were separated, and the organic layer obtained was partially reduced by distillation under vacuum to obtain a reaction mass. To the reaction mass was then added 375.6 ml of ethanol and 250 ml of thionyl chloride in 6 hours. The mixture was maintained at room temperature for 12 hours until product formation is completed. The solvent used was recovered by distillation under vacuum at temperature of about 60-70° C. to obtain 371.57 g of ethyl (2S)-4-chloro-2-[(ethoxycarbonyl)amino]butanoate having wt/wt purity of 93.9%. Overall Yield 73.14%

### Example 3: Preparation of Ethyl (2S)-4-chloro-2-[(ethoxycarbonyl)amino]butanoate-[Compound of Formula Ia]from O-Acetyl Homoserine

A mixture of 300 g of O-acetyl Homoserine and 240.6 ml of 30% HCl was heated to 80-90° C. Then the aqueous layer was distilled under vacuum at 60-70° C. till moisture content of reaction mass was below 8-10%. After completion of reaction, to the mixture was added 600 ml of water. The pH of aqueous layer obtained was adjusted to 5.0 to 5.5 using aqueous solution of sodium carbonate and subsequently 480 ml of ethylene chloride was added to it. To this mixture was then added sodium carbonate solution (prepared from 232 g of sodium carbonate and 600 ml of water) and a solution of ethyl chloroformate (prepared from 218 g 191.2 ml of ethyl chloroformate and 300 ml of ethylene dichloride) at 0 to −5° C. in 3 to 4 hours. The layers were separated, and the organic layer obtained was partially reduced by distillation under vacuum to obtain a reaction mass. To the reaction mass was then slowly added 158.5 ml of ethanol and 197 ml of thionyl chloride in 6 hours. The mixture was maintained at room temperature for 12 hours until product formation is completed. The solvent used was recovered by distillation under vacuum to obtain 384.76 g of ethyl (2S)-4-chloro-2-[(ethoxycarbonyl)amino]butanoate having wt/wt purity of 95.8%. Overall Yield 77.60%

### Example 4: Preparation of Ethyl (2S)-4-chloro-2-[(ethoxycarbonyl)amino]butanoate-[Compound of Formula Ia]from L-Homoserine

A mixture of 72 g of L-homoserine and 81.3 ml of 30% HCl was heated to 75-85° C. Then the acidic water was distilled under vacuum at 50-60° C. To the slurry obtained was added 75 ml cyclohexane and the water was removed azeotropically at 70-75° C. till moisture content of reaction mass was below 8%. After completion of the reaction, to the mixture was added 200 ml of water. To the mixture was then added 200 ml of ethylene chloride followed by addition of sodium carbonate solution (prepared from 75.5 g of sodium carbonate and 250 ml of water) and a solution of ethyl chloroformate (prepared from 63 ml of ethyl chloroformate and 100 ml of ethylene dichloride) at 0 to −5° C. at pH 5.5 to 7.5. The layers were separated, and the organic layer obtained was partially reduced by distillation under vacuum to obtain a reaction mass. To the reaction mass was then slowly added 46 g 58.3 ml of ethanol and 71.4 ml of thionyl chloride in 5-6 hours. The mixture was maintained at room temperature for 6-10 hours, the mixture was then washed with water and saturated aqueous solution of sodium bicarbonate. The solvent used was recovered by distillation under vacuum to obtain 130 g of ethyl (2S)-4-chloro-2-[(ethoxycarbonyl)amino]butanoate having % wt/wt purity of 92.58%. Overall Yield 83.7%

### Comparative Example 1: Preparation of Ethyl (2S)-4-chloro-2-[(ethoxycarbonyl)amino]butanoate [Compound of Formula Ia]

### Step 1: Preparation of (3S)-3-aminodihydrofuran-2(3H)-one hydrochloride-[Compound of Formula IVb]

To 900 g ml of water was added 300 g of L-Methionine, 304 ml of ethyl chloroacetate, 23 g of tetrabutylammonium bromide and the mixture was heated to 90° C. for 12 hours. After completion of reaction, the layers were separated, and the aqueous layer was extracted with 480 ml of dichloromethane. To the aqueous layer was then added 30% aqueous hydrochloric acid and the mixture was stirred for 5 hours, further the water in aqueous layer was distilled out completely to obtain crude product. To the crude product was added 600 ml ethanol and this suspension was cooled to 0° C., the solid precipitated out was filtered and dried to get 226 g of (3S)-3-aminodihydrofuran-2(3H)-one hydrochloride.

### Step 2: Preparation of Ethyl [(3S)-2-oxotetrahydrofuran-3-yl]carbamate-[Compound of Formula VIa]

In 452 g ml of water was added 226 g of (3S)-3-aminodihydrofuran-2(3H)-one hydrochloride and 501 ml of ethyl acetate and the mixture was cooled to −5 to 0° C. To this mixture was then added simultaneously aqueous sodium carbonate solution (prepared from 213.1 g of sodium carbonate and 452 ml of water) and a solution of ethyl chloroformate (prepared from 175.7 ml of ethyl chloroformate and 226 ml of ethyl acetate) at 0 to −5° C. in 3 to 4 hours under stirring. The temperature of the mixture was increased to 25-30° C. and the layers were separated. The aqueous layer was extracted with ethyl acetate. The organic layers obtained were combined and the solvent was distilled off to get crud product. To the crude product was added 684 ml of hexane, mixture was stirred, solid product was then filtered out and dried to get 285.6 g of ethyl [(3S)-2-oxotetrahydrofuran-3-yl]carbamate.

### Step 3: Preparation of Ethyl (2S)-4-chloro-2-[(ethoxycarbonyl)amino]butanoate-[Compound of Formula Ia]

To a mixture of 429.4 ml of dichloromethane and 370 ml of ethanol and 285.6 g of ethyl [(3S)-2-oxotetrahydrofuran-3-yl]carbamate was added dropwise 241 ml of thionyl chloride. The resultant mixture was stirred and maintained for 12 hr. After completion of reaction, the mixture was heated to 70-80° C. and the organic solvents were distilled out under vacuum. The reaction mass obtained was extracted with 429.4 ml of dichloromethane and 285.6 g ml of water, the organic layer was separated and distilled off to get 368 g of ethyl (2S)-4-chloro-2-[(ethoxycarbonyl)amino]butanoate having 93% wt/wt purity. Overall Yield 58.8%.

1. A process for preparation of a compound of formula (I) 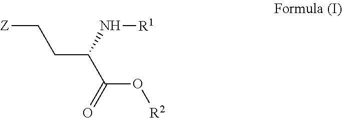
wherein, R¹ is an amino protecting group, R² is a carboxylic acid protecting group; and Z is halogen or a hydroxy group;
the process comprising converting L-homoserine lactone of formula (IV) or its salt, to compound of formula (I): 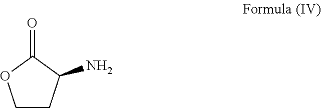
wherein the compound of formula (IV) is substantially free of a compound of formula (V): 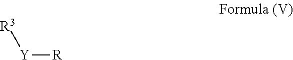
wherein:
R is hydrogen, alkyl, aryl, acyl, alkylsulfonate esters, alkylnitrate ester, alkylphosphoryl, or alkylsilyl;
Y is selected from O, N, and S, and
R³ is hydrogen or R⁴—O—C(O)—CH₂—, wherein R⁴ is selected from hydrogren, and a C₁-C₅ alkyl group.

2. The process as claimed in claim 1, wherein the compound of formula (IV) substantially free of a compound of formula (V) is prepared by removing the compound of formula (V) from the reaction product obtained by reacting e compound of formula (II) with a compound of formula (III): 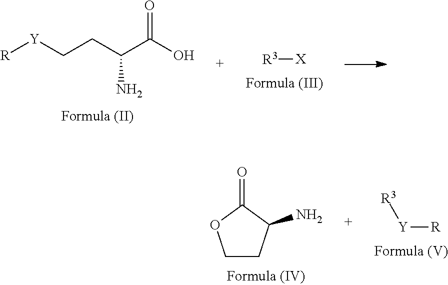
wherein,
R is hydrogen, alkyl, aryl, acyl, alkylsulfonate esters, alkylnitrate ester, alkylphosphoryl, or alkylsilyl;
R³ is hydrogen or R⁴—O—C(O)—CH₂—
R⁴ is selected from hydrogen, and a C₁ to C₅ alkyl group;
X is halogen, —HSO₄, or —H₂PO₄, and
Y is selected from O, N and S.

3. The process as claimed in claim 1, wherein the formula (I) represents a compound
wherein said amino protecting group is selected from an acyl group C(═O)A, wherein A is OR′ or R′, wherein R′ is a substituted or unsubstituted C₁ to C₁₀ alkyl group, a substituted or unsubstituted C₁ to C₁₀ alkenyl group, a substituted or unsubstituted C₁ to C₁₀ alkynyl group, a substituted or unsubstituted C₃ to C₁₀ cycloalkyl group, a substituted or unsubstituted C₆ to C₂₀ aryl group, or a substituted or unsubstituted C₂ to C₁₀ heteroaryl group; urea; urethane; nitroso; nitro; sulphenyl; sulphonyl; sulfonic acid; and trialkylsilyl; and
wherein said carboxylic acid protecting group is selected from a substituted or unsubstituted C₁ to C₁₀ alkyl group, and a substituted or unsubstituted C₆ to C₂₀ aryl group.

4. The process as claimed in claim 1, wherein the formula (I) represents a compound wherein said amino protecting group is selected from an acyl group C(═O)A, wherein A is OR′ or R′, wherein R′ is a substituted or unsubstituted C₁ to C₅ alkyl and wherein said carboxylic acid protecting group is a substituted or unsubstituted C₁ to C₅ alkyl group; and wherein Z is halogen.

5. The process as claimed in claim 2, wherein the formula (II) represents (i) a compound wherein Y is selected from O, N or S; R is hydrogen, C₁ to C₅ alkyl, or an acyl group of formula —C(═O)R′, and wherein R′ is C₁ to C₅ alkyl, (ii) a compound, wherein Y is S and R is C₁ to C₅ alkyl, (iii) L-methionine, or (iv) a compound wherein Y is O; and R is hydrogen or an acyl group of formula —C(═O)R′, wherein R′ is C₁ to C₅ alkyl.

6. The process as claimed in claim 5, wherein the formula (II) compound is L-homoserine, O-acetyl L-homoserine, or a combination thereof.

7. The process as claimed in claim 2, wherein the formula (III) represents (i) a compound wherein R³ is hydrogen or R⁴—O—C(O)—CH₂—, R⁴ is selected from hydrogen and a C₁ to C₅ alkyl group; and X is halogen; (ii) a compound wherein R³ is hydrogen and X is halogen, or (iii) a compound wherein R³ is R⁴—O—C(O)—CH₂—, R⁴ is selected from hydrogen and C₁ to C₅ alkyl group; and X is halogen.

8. The process as claimed in claim 2, wherein the reaction between compound (II) and (III) is carried out in the presence of an acid catalyst when the formula (III) represents a compound wherein R³ is R⁴—O—C(O)—CH₂, R⁴ is selected from hydrogen and C₁ to C₅ alkyl group; and X is halogen.

9. The process as claimed in claim 8, wherein said acid catalyst is selected from p-toluene sulfonic acid, methane sulfonic acid, benzene sulfonic acid, hydrochloric acid, phosphoric acid, sulfuric acid, trichloroacetic acid, and trifluoroacetic acid.

10. The process as claimed in claim 8, wherein the reaction of the compound of formula (II) with the compound of formula (III) is carried out in the presence of a solvent, and the solvent is selected from water, an organic solvent selected from C₁-C₅ alcohols, and a mixture thereof.

11. The process as claimed in claim 1, wherein the L-homoserine lactone of formula (IV) is in form of a salt.

12. The process as claimed in claim 2, wherein the compound of formula (V) is removed from the reaction mixture by distillation, azeotropic distillation, or solvent extraction followed by distillation or chemical treatment.

13. The process as claimed in claim 1, wherein: the process for preparation of the compound of formula (I) further comprises protection of an amino group of the L-homoserine lactone of formula (IV) or its salt, to obtain a compound of formula (VI) followed by conversion to a compound of formula (I).

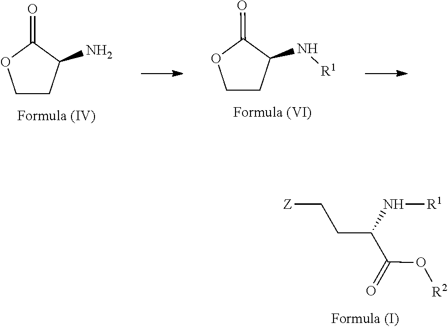

14. The process as claimed in claim 13, wherein the compound of formula (VI) is converted to the compound of formula (I) by treatment with a halogenating agent and an alcohol.

15. The process as claimed in claim 14, wherein said alcohol is C₁-C₈ alcohol, and said halogenating agent is selected from the group consisting of chlorine, phosphorus trichloride, phosphorus pentachloride, phosphorus oxychloride, thionyl chloride, sulfuryl chloride, oxalyl chloride, sulfonyl chloride, acetyl chloride, phosgene, bromine, phosphorus tribromide, boron tribromide, iodine, phosphorus triiodide, N-chlorosuccinimide, and N-bromosuccinimide.

16. The process as claimed in claim 15, wherein said process proceeds without isolation or purification of the homoserine lactone of formula (IV) or its salt; and the compound of formula (VI).

17. The process as claimed in claim 1, wherein the process proceeds without isolation or purification of an intermediates formed in any of the steps of the process.

18. A process for preparing L-glufosinate or its salts, wherein the process comprises preparing a compound of formula (I) 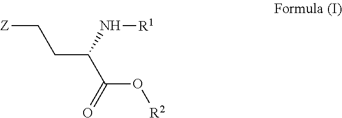
wherein, R¹ is an amino protecting group, R² is a carboxylic acid protecting group, and Z is halogen or a hydroxy group;
converting n L-homoserine lactone of formula (IV) or its salt, to compound of formula (I): 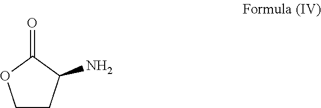
wherein the compound of formula (IV) is substantially free of a compound of formula (V): 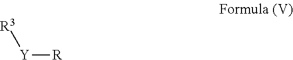
wherein:
R is hydrogen, alkyl, aryl, acyl, alkylsulfonate esters, alkylnitrate ester, alkylphosphoryl, or alkylsilyl;
Y is selected from O, N, and S; and
R³ is hydrogen or R⁴—O—C(O)—CH₂—, wherein R₄ is selected from hydrogen, and a C₁ to C₅ alkyl group.

19. The process as claimed in claim 1, wherein the yield of the compound of formula (I) is enhanced by at least 5%.
